(19) United States
(12) Patent Application Publication
(10) Pub. No.: US 2007/0078396 A1
(43) Pub. Date: Apr. 5, 2007

(54) Tunneler for use dual lumen tip catheter

(76) Inventors: Kristin Feeley, Hingham, MA (US); David G. Quinn, Grayslake, IL (US)

Correspondence Address: FAY KAPLUN & MARCIN, LLP, 15O BROADWAY, SUITE 702, NEW YORK, NY, 10038, US

(21) Appl. No.: 11/228,714

(22) Filed: Sep. 16, 2005

(51) Int. Cl. A61M 5/178

(52) U.S. Cl. 604/164.01

(57) ABSTRACT

A tunneler for a dual lumen tip catheter, comprises an elongated body extending from a distal tissue-penetrating end to a proximal, catheter-coupling end, the catheter coupling end including a catheter tip receiving portion including a cutout sized and shaped to receive a dual lumen tip of a catheter to be coupled thereto and a proximally extending lumen-mating projection oriented so that, when inserted into a lumen of the catheter, the dual lumen tip of the catheter is received in the cutout in an orientation relative to an elongated body of the catheter substantially the same as when the dual lumen tip and the elongated body of the catheter are in an unstressed state.

### BACKGROUND OF THE INVENTION

Catheters are routinely used to form a semi-permanent path into the body through which fluids can pass to and from target sites eliminating the need for repeated insertions of needles, etc.

Some vascular catheters, such as peripherally inserted central catheters (PICC's) and dialysis catheters are not generally inserted through the skin at locations adjacent to the site of entry into a target blood vessel. Instead, a tunnel is formed under the skin between a location at which the catheter enters a target blood vessel and a location at which the catheter penetrates the skin. The length of the tunnel connecting the two locations may differ depending on the purpose of the catheter and the anatomy of the patient.

The need to minimize injury to surrounding tissues often complicates the procedure for forming the tunnel. Because the ends of these catheters are typically too large to form the tunnel without excessive trauma, a separate device called a tunneler is typically connected to the catheter. The tunneler is an elongated device with a tapered tip designed to pass through the tissue making a path along which the catheter may pass without damaging the surrounding tissue. A second incision is then made near the location at which the catheter is to enter the target blood vessel to facilitate removal of the tunneler and insertion of the catheter into the target blood vessel.

### SUMMARY OF THE INVENTION

In one aspect, the present invention is directed to a tunneler for a dual lumen tip catheter, comprising an elongated body extending from a distal tissue-penetrating end to a proximal, catheter-coupling end, the catheter coupling end including a catheter tip receiving portion including a cutout sized and shaped to receive a dual lumen tip of a catheter to be coupled thereto and a proximally extending lumen-mating projection oriented so that, when inserted into a lumen of the catheter, the dual lumen tip of the catheter is received in the cutout in an orientation relative to an elongated body of the catheter substantially the same as when the dual lumen tip and the elongated body of the catheter are in an unstressed state.

### BRIEF DESCRIPTION OF THE DRAWINGS

FIG. 1 shows a side elevation view of an embodiment of a metal tunneler according to the present invention;

FIG. 2 shows a top plan view of an embodiment of a metal tunneler according to the present invention;

FIG. 3 shows a side elevation view of an embodiment of a plastic tunneler according to the present invention;

FIG. 4 shows a top plan view of an embodiment of a plastic tunneler according to the present invention;

FIG. 5 shows a detailed view of a proximal end of an embodiment of the tunneler according to the present invention;

FIG. 6 shows a detailed view of a proximal end of the tunneler with a sheath according to the present invention;

FIG. 7 shows a detailed view of a proximal end of another embodiment of the tunneler according to the present invention;

FIG. 8 shows a detailed view of a proximal end of a further embodiment of the tunneler according to the present invention;

FIG. 9 shows a detailed view of a proximal end of a different embodiment of the tunneler according to the present invention;

FIG. 10 shows a detailed view of a proximal end of yet another embodiment of the tunneler according to the present invention;

FIG. 11 shows a detailed view of a proximal end of an embodiment of the tunneler according to the present invention, having a lip;

FIG. 12 shows a perspective view of a sheath for use with any of the tunnelers according to the present invention; and

FIG. 13 shows a cross-sectional side view of the sheath of FIG. 12.

### DETAILED DESCRIPTION

The present invention may be further understood with reference to the following description and the appended drawings, wherein like elements are referred to with the same reference numerals. The invention is related to tunneler devices used in conjunction with devices used to introduce and/or remove therapeutic compounds from the body. More specifically, the invention is related to a novel construction for a catheter having a dual lumen tip.

Catheters are basically flexible tubes formed by an outer shell which defines an inner lumen which may serve as a fluid conduit. Various fittings or connections may be used at the proximal end to connect the catheter to one or more additional devices. The outer shell is typically formed of a material selected to be impermeable to the fluid flowing therein with the length and diameter of the catheters varying considerably depending on the application. Generally, catheters are formed of flexible, biocompatible materials (e.g., polymeric materials) to reduce discomfort during insertion and during subsequent movement of the patient.

As described above, tunnelers are often connected to catheters to aid in creating a path between a location at which the catheter penetrates the skin and a location at which the catheter is to enter a target vessel. A conventional tunneler is commonly connected to the catheters by inserting a protrusion of the tunneler into a lumen of the catheter with outer surfaces of the tunneler extending around the outer surfaces of the catheter. Most conventional tunnelers increase in diameter toward their proximal ends (i.e., the ends at which they are coupled to catheters). In addition, the insertion of a tunneler barb into the distal end of a catheter may increase the diameter of the distal end of the catheter. However, the lumens of dual lumen tip catheters which are often used in procedures in which blood is removed and inserted simultaneously are partially hidden by the dual lumen tip making it difficult to insert the projection of a conventional tunneler therein.

In kidney dialysis, for example, a single catheter may be used to remove blood from the a blood vessel for treatment while simultaneously reintroducing treated blood to the vessel. Although the same catheter is used for the removal and reintroduction of blood, it is often desirable to separate these two flows of blood to, for example, prevent the removal for treatment of blood which has already been treated. Dual lumen tip catheters are designed to maintain inlet and outlet flows separate from one another.

Dual lumen tips often extend radially outward beyond an outer surface of other portions of the catheter body, increasing the cross sectional area of the catheter that has to be pushed through the subcutaneous tunnel. The dual lumen tip may also extend radially inward toward a centerline of the catheter so that it is pushed further radially outward when a conventional tunneler is inserted into the lumen.

Many dual lumen tip catheters have a distal, lumenal opening separated by a distance from the distal end of the catheter itself. In the case of dual lumen catheters, the distal openings of the two lumens are also separated longitudinally from one another. The distal region of the dual lumen tip may partially block the lumen openings, due to its curved shape. Because of the configuration of the dual lumen tip, when a standard tunneler is inserted, it pushes against the dual lumen tip causing the dual lumen tip to ride over the body of the tunneler and extend radially outward from the catheter's centerline. This increases the profile size of the tunneler/catheter combination and impedes tunneling. Additionally, the dual lumen tip may flex or bend backwards during the tunneling operation, as the dual lumen tip catches against the surrounding tissue increasing the risk of damage to the catheter and/or the surrounding tissues.

The tunneler according to embodiments of the present invention includes a proximal cutout section enabling it to mate with the distal end of a dual lumen tip catheter with the dual lumen tip laying in a streamlined position along a body of the tunneler reducing the size of the tunnels and easing the passage of the tunneler/catheter combination through the tissue. The proximal end of the tunneler according to the present invention thus forms a conformal connector shaped to provide a streamlined, low profile transition between the dual lumen tip catheter and the tunneler.

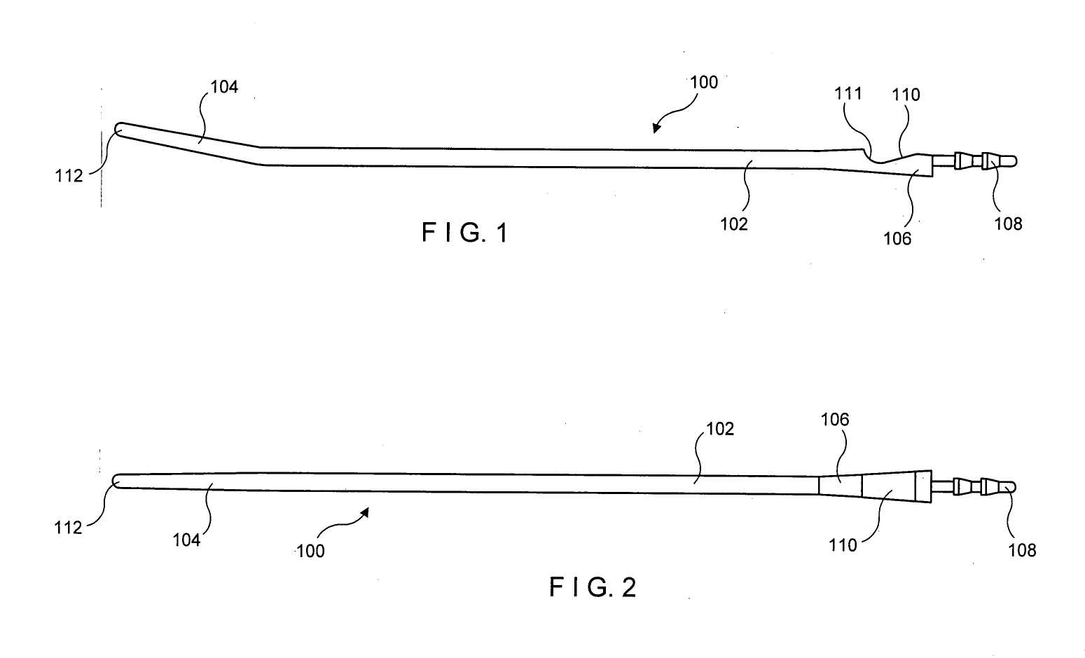

FIGS. 1 and 2 show an exemplary embodiment of a tunneler according to the present invention. The tunneler 100 is shown in a side elevation view (FIG. 1) and in a top plan view (FIG. 2). The tunneler 100 may be formed of a metal compatible with biological uses and includes an elongated body 102 that extends for a length which may be selected to be substantially equal to a length of a subcutaneous tunnel to be formed. For example, the tunneler 100 may be formed of stainless steel that is machined in a conventional manner to the desired shape. A distal portion 104 may be curved or tilted from the axis of the elongated body 102, to facilitate extraction of the tunneler 100 from the subcutaneous tunnel, through an incision in the skin. The tip 112 is preferably tapered so that a diameter of a distal end of the tip 112 is slightly reduced with respect to that of a central portion of the elongated body 102. The tip 112 may also be rounded or otherwise shaped to facilitate blunt dissection.

The exemplary tunneler 100 comprises a proximal portion 106 designed to securely and releasably attach to a dual lumen tip catheter. As will be described in greater detail below, the proximal portion 106 includes a protrusion 108 which preferably includes barbs 109 (or other suitable structure) to secure the protrusion 108 within a lumen of a catheter and a cutout 110 with a shaped surface 111 that conforms to a shape of a radially inward surface of the dual lumen tip 210 (i.e., the outer surface of the portion of the dual lumen tip 210 which, due to the offset of this tip from the axis of the catheter 202, is closer to the axis) to form a streamlined and secure connection with the catheter. For example as shown in FIG. 2, the proximal portion 106 of the tunneler 100 includes an expanded diameter section 107 that flares gradually, radially outward in a distal to proximal direction. According to this embodiment, the cutout 110 is formed in this expanded diameter section 107 so that, when a catheter is received within the cutout 110 with the protrusion 108 inserted into a lumen opening of the catheter, a portion of an outer surface of the catheter on one side of the lumen opening lies flush with a surface of the proximal-most part of the expanded diameter section 107 while an outer surface of the catheter on the opposite side of the lumen opening lies flush with a surface of the expanded diameter section 107 adjacent a distal end of the cutout 110 with the dual lumen tip of the catheter received in the cutout 110. Those skilled in the art will recognize that the outer surface of the catheter need not be flush with or radially within an outer diameter of the tunneler when connected thereto especially if used in conjunction with a sheath as described below. Rather, it is important only that the cutout design minimize and streamline the profile of the combined catheter and tunneler.

In use, the protrusion 108 is inserted into a lumen of a dual lumen tip catheter via a distal lumen opening to connect the tunneler 100 to the dual lumen tip catheter. A sheath (described in more detail below) may then be placed over the proximal portion 106 of the tunneler 100 and over a distal portion of the catheter (e.g., covering the distal lumen opening(s) thereof). The tunneler 100 is then inserted through an incision at the catheter entry site and passed through the tissue dragging the catheter along after it to a location near the site at which the catheter is to enter a target anatomical structure (e.g., a blood vessel). The tunneler 100 is then removed from the catheter and withdrawn from the body via an incision at this location and the catheter is inserted into the target anatomical structure.

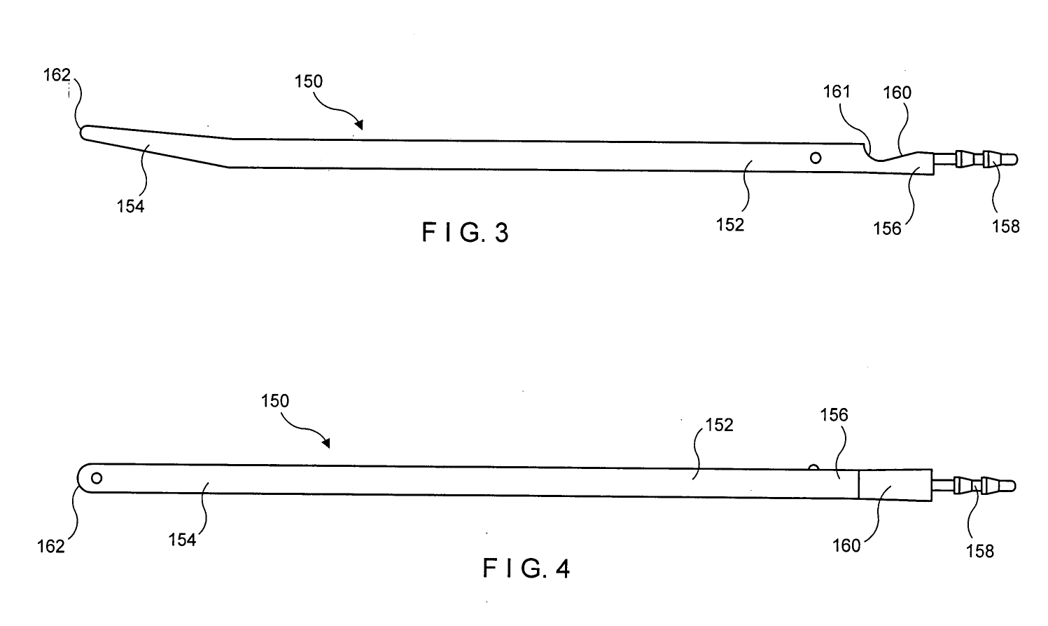

FIGS. 3 and 4 show a tunneler 150 according to a second embodiment of the invention. The tunneler 150 is optimized for manufacture from a plastic material, such as, a biocompatible polymer as would be understood by those skilled in the art. For example, Delrin may be used in a conventional molding process to form the tunneler 150 comprising an elongated body 152 that extends for a length selected to substantially equal a length of a tunnel to be formed. A curved distal end 154 thereof includes a tip 162 shaped to facilitate the penetration of tissue. A cutout region 160 with a shaped surface 161 is provided, to substantially match the shape of a portion of the dual lumen tip to be received therein. The elongated body 152 of the tunneler 150 includes a cutout 160 similar to the cutout 110 of the tunneler 100 for receiving the distal tip of a radius catheter (not shown). The arrangement of the cutout 160 and the contoured surface 161 thereof of this embodiment is similar to that of the tunneler 100 so that, when a protrusion 158 extending from the proximal end of the tunneler 150 is received in the lumen of a dual lumen tip catheter, the distal tip of the catheter fits within the cutout 160 with outer surfaces of the catheter substantially aligning with an outer surface of the proximal end of the elongated body 152 adjacent to the protrusion 158 and an outer surface of the elongated body 152 adjacent to a distal end of the cutout 160.

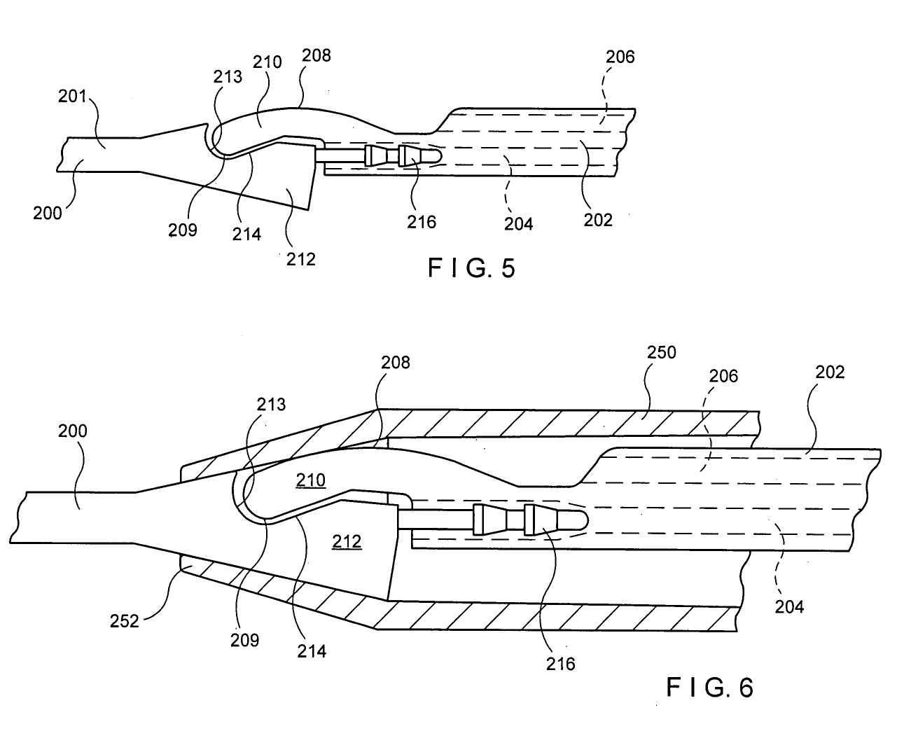

A more detailed view of a proximal end of a tunneler 200 according to a further embodiment of the present invention is shown in FIG. 5. The tunneler 200 comprises a conformal connector having features according to the present invention that optimize it for use with a dual lumen tip catheter 202. A typical dual lumen tip catheter 202 which is to be used with the tunneler 200 comprises, for example, first and second lumens 204, 206 for removing blood from and returning blood to a target blood vessel. The lumen 206 for removing blood is referred to as the arterial lumen and the lumen 204 for returning blood is referred to as the venous lumen which opens closer to the distal tip of the catheter 202. A dual lumen tip 210 is formed at the distal end of the catheter 202, to separate the distal openings of the lumens 204, 206 from one another to limit the recirculation of blood between the outlet of the venous lumen 204 and the inlet of the arterial lumen 206.

As shown in FIG. 5, the tunneler 200 comprises a protrusion 216 sized to fit into the venous lumen 204 with one or more barbs 216′ formed on the protrusion 216 to secure the protrusion 216 within the lumen 204. As would be understood by those skilled in the art, the barbs 216′ which frictionally engage an inner wall of the lumen 204 to secure the tunneler 200 to the catheter 202 may be replaced by any other suitable anchoring structure. The protrusion 216′ preferably extends from a proximal end 212 of the tunneler 200 so that, when inserted into the opening of the lumen 204, the dual lumen tip 210 extends distally past the proximal end 212 into a cutout 213 described in more detail below.

Similarly to the tunnelers 100 and 150 described above, the proximal end 212 of the tunneler 200 includes a cutout 213 which receives the dual lumen tip 210 without deforming the dual lumen tip 210 or pushing it radially outward. The cutout 213 which is formed in an increased diameter section 215 includes a contoured surface 214 which, when the protrusion 216 is received in the lumen 204, substantially mirrors a shape of the lower surface 209 of the dual lumen tip, so that the two parts can fit like pieces of a puzzle. After the tunneler 200 is attached to the catheter 202, the dual lumen tip 210 is seated in place along the proximal end 212, forming a streamlined, low profile connection.

Since the catheters and the dual lumen tips are made of elastic materials, the dual lumen tips may be deflected slightly while the protrusion of the tunneler is pushed into the lumen of the catheter and the dual lumen tip is moved into the cutout to form the connection. For example, the dual lumen tip 210 bends to pass over high points of the contoured surface 214, and then snaps back to its normal, unstressed shape when the connection is complete. The contoured surface 214 is preferably shaped to form a substantially continuous, smooth transition between the outer surface of the tunneler body 201 and the upper surface 208 of the dual lumen tip 210. For example, the proximal end 212 of the tunneler 200 and the contoured surface 214 preferably have dimensions that increases gradually from a diameter of the tunneler body 201 to a maximum diameter of the catheter 202. This minimizes the risk of snagging or catching of tissue as the tunneler/catheter combination passes through the tissue. Because many catheters such as the catheter 202 increase in diameter moving proximally away from their distal ends and to accommodate an increase in catheter diameter associated with the insertion of the protrusion of the tunneler therein, increasing the diameter of the proximal end of the tunneler (e.g., tunneler 200) to a level equal to the maximum diameter of the catheter facilitates tunneling.

The exemplary embodiments of the tunneler according to the present invention described herein provide a more streamlined transition between the catheter and the tunneler facilitating subcutaneous dissection and reducing the trauma associated therewith by minimizing an overall maximum diameter of the joined tunneler/catheter combination. The mechanical connection between catheter and tunneler is also strengthened by the matching of the shape of the contoured surface of the tunneler to the shape of the dual lumen tip. Thus, the catheter may be drawn through the subcutaneous tissue by pulling on the distal portion of the tunneler without prematurely separating the two components from one another.

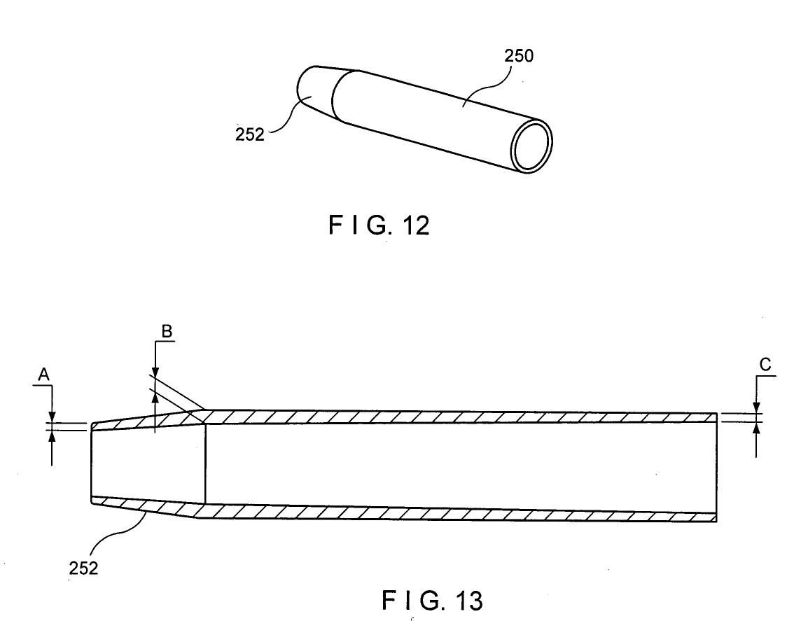

FIG. 6 shows a tunneler 200 as described above in conjunction with a sheath or oversleeve 250 which is placed around a proximal portion of the tunneler 200 and a distal portion of the catheter 202. The sheath 250 comprises a shell-like elongated body with a diameter sufficient to encompass the connection between the dual lumen tip 210 and the proximal end 212 of the tunneler 200 without substantially increasing the diameter of the connection. That is, the sheath preferably increases an outer diameter of the tunneler/catheter combination by only twice the thickness of the sheath material. The sheath may, for example, be formed of an injection molded polymer such as, for example, high density polyethylene. As shown in FIGS. 12 and 13, the sheath 250 includes a tapered distal end 252. Furthermore, the thickness of the sheath 250 may vary along the length thereof. As shown in FIG. 13, the thickness decreases from a maximum near a proximal end of the tapered distal end 252 toward each of the ends of the sheath. For example, a thickness of the ends of the sheath may be between 20 and thirty thousandths of an inch while the maximum thickness is between 35 and 45 thousandths of an inch.

After the tunneler 200 and the catheter 202 have been connected to one another, the sheath 250 may be slid over the distal end of the tunneler 250 and drawn proximally thereover until the tapered distal end 252 of the sheath 250 encounters the increased diameter section 215. The distal end 252 of the sheath 250 is preferably sized so that it is unable to pass over this increased diameter section 215 and, when the distal end 252 of the sheath 250 is received therearound, the sheath 250 extends proximally over the increased diameter section 215 and over the distal portion of the catheter 202. When in this position, the sheath 250 preferably covers the distal openings of both the venous and arterial lumens 204, 206. The sheath 250 also applies an additional interference fit and force to hold the catheter 202 in place on the tunneler 200 during use.

The addition of the sheath 250 may further reduce trauma to the surrounding tissue by preventing the surrounding tissue from catching (e.g., in the areas between the tunneler 200 and the catheter 202). The sheath 250 preferably fits snugly over the connection of the catheter 202 and the tunneler 200, so that it is not easily dislodged during use and increases the stability of the connection. In addition, the sheath 250 may be useful with split tip catheters as it will help maintain the arterial and venous lumens together during tunneling. The sheath 250 is also helps reduce the possibility of contaminants being introduced into the lumens during tunneling.

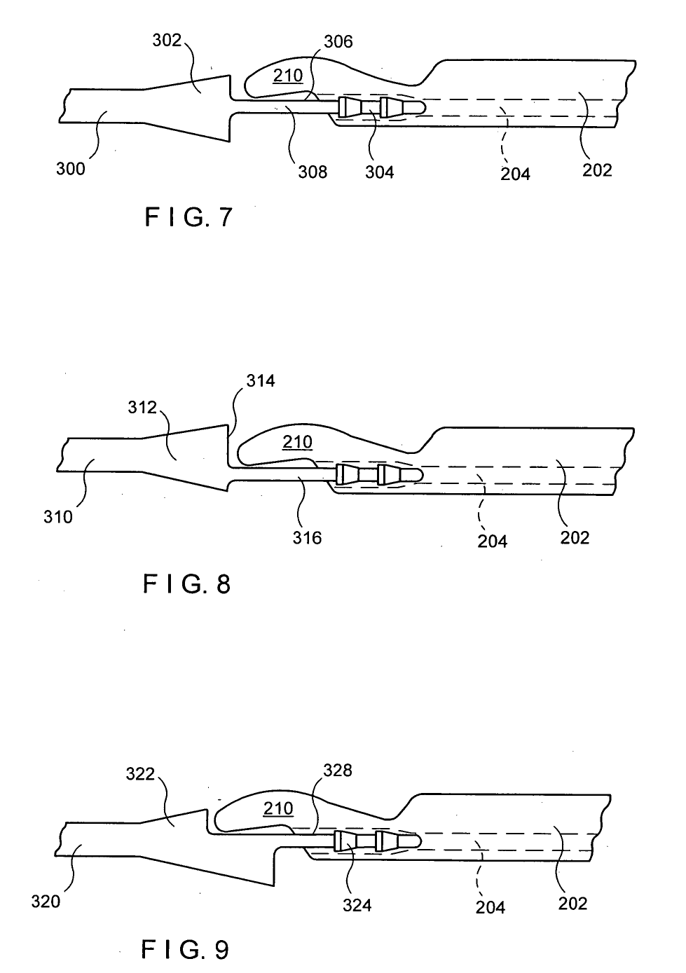

A tunneler 300 according to a still further embodiment of the present invention is shown in FIG. 7. The tunneler 300 comprises a proximal portion 302 that forms a stop for the dual lumen tip 210 of the catheter 202. A stem 308 of the tunneler 300 includes one or more barbs 304 and has an increased length compared to the projections of conventional tunnelers. The increased length of the stem 308 more effectively resists deflection of the dual lumen tip 210 away from the axis of the catheter 202 as the all of the dual lumen tip 210 remains proximal of the proximal end of the proximal portion 302 of the tunneler 300. For example, if a barb portion 304 is inserted into the venous lumen 204 with a length of the stem 308 extending between the distal opening of the lumen and the proximal end of the proximal portion 302 substantially equaling a length of the dual lumen tip 210, the dual lumen tip 210 will lie along an upper surface 306 of the elongated stem 308 with a distal end of the dual lumen tip 210 adjacent to the proximal end of the proximal portion 302. As the complex shape of the cutout is eliminated in the tunneler 300, this device is simpler to manufacture than the previously described embodiments.

FIG. 8 shows a tunneler 310 according to a variation of the exemplary embodiment described above in regard to FIG. 7. The tunneler 310 comprises a proximal portion 312 having a barb 316 offset with respect to a centerline of the tunneler 302 so that, when inserted in the venous lumen 204, the catheter 202 is shifted off-center drawing the dual lumen tip toward the centerline of the tunneler 310 and reducing the profile of the tunneler/catheter combination. That is, the dual lumen tip 210 fits between a stop surface 314 of the proximal portion 312 and the offset barb 316, so that it does not protrude radially outside the proximal portion 312. In one exemplary embodiment, the barb portion 316 is offset radially from the tunneler's centerline by a distance comparable to a radial width of the dual lumen tip 210.

In one exemplary embodiment of the tunneler according to the present invention, the upper surface of the tunneler's proximal portion can be made flat rather than contoured to closely match the shape of the dual lumen tip. In this embodiment, shown in FIG. 9, the tunneler 320 has a proximal end 322 comprising a flat upper surface 328 over which can slide the dual lumen tip 210. The barb portion 324 is designed to fit in the venous lumen 204, to provide a releasable connection between the catheter 202 and the tunneler 320. This exemplary embodiment avoids the complication of manufacturing a contoured upper surface of the tunneler 320 that is tailored to one specific dual lumen tip catheter. However, the present connection results in a greater profile than would be the case if a contoured upper surface 328 was used.

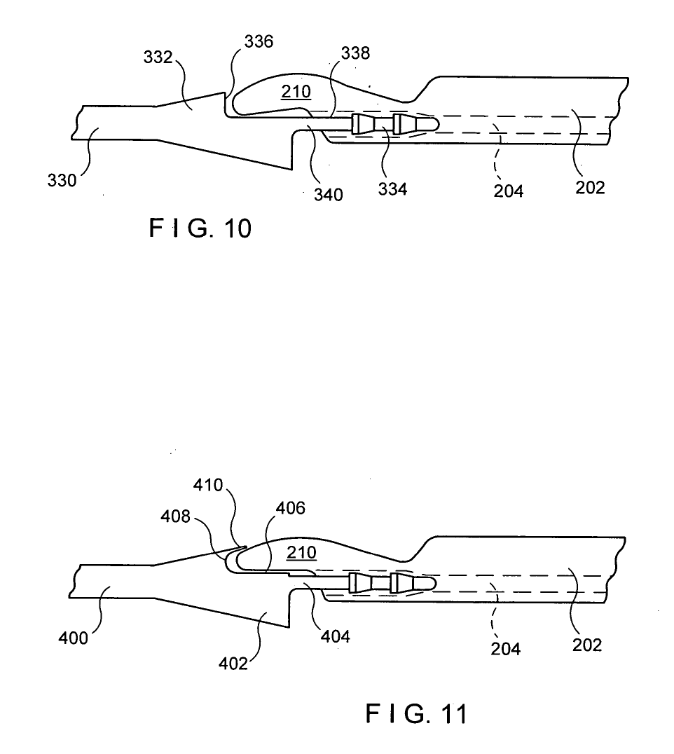

A further refinement of the tunneler connection described above is shown in FIG. 10. Here, the tunneler 330 includes a barb element 334 with an offset barb shaft 340 which further reduces the profile of the connection between the catheter 202 and the tunneler 330. The proximal portion 332 of the tunneler 330 includes an upper surface 338 that is substantially flat and which is generally shaped to provide room for the dual lumen tip 210 to slide thereover without being deflected when the barb element 334 is received within a lumen of the catheter 202. As in the embodiments described above, the proximal portion 332 of the tunneler 330 flares outward to smooth the transition between the elongated body of the tunneler 330 and increased diameter of the assembly including the dual lumen tip 210 and the barb element 334, facilitating tunneling through tissue by maintaining the dual lumen tip 210 substantially axially aligned so that it is not bent outward to catch on surrounding tissues.

As shown in FIG. 11, a tunneler 400 according to another exemplary embodiment the present invention includes a proximal portion 402 with a barb element 404 sized and positioned for insertion into the venous lumen 204 of a catheter 202. The tunneler 400 includes a proximal portion 402 with a dual lumen tip receiving surface 406 shaped to received the radially interior surface of the dual lumen tip 210 of the catheter 202. As with several of the above-described embodiments, the dual lumen tip receiving surface 406 may be substantially flat or contoured in a shape corresponding to the shape of the radially interior surface of the dual lumen tip 210 of the catheter 202 to provide sufficient space for the dual lumen tip 210 to lie substantially flat against the proximal portion 402, without being deflected from the orientation it would assume relative to the rest of the catheter 202 when in an unstressed state. A lip 410 extends proximally from the tunneler 400, partially covering the cutout 409 to assist in retaining the dual lumen tip 210 in place within the cutout 409. Principally, the lip 410 prevents the dual lumen tip 210 from projecting radially out from the tunneler 400 minimizing the risk of the dual lumen tip 210 snagging on the surrounding tissue or being bent backwards during insertion.

The present invention has been described with reference to specific embodiments, and more specifically to a tunneler for use with dialysis catheters and PICC catheters. However, other embodiments maybe devised that are applicable to other medical devices and procedures, without departing from the scope of the invention. Accordingly, various modifications and changes may be made to the embodiments, without departing from the broadest spirit and scope of the present invention as set forth in the claims that follow. The specification and drawings are accordingly to be regarded in an illustrative rather than restrictive sense.

What is claimed is:

1. A tunneler for a dual lumen tip catheter, comprising an elongated body extending from a distal tissue-penetrating end to a proximal, catheter-coupling end, the catheter coupling end including:
a catheter tip receiving portion including a cutout sized and shaped to receive a dual lumen tip of a catheter to be coupled thereto; and
a proximally extending lumen-mating projection oriented so that, when inserted into a lumen of the catheter, the dual lumen tip of the catheter is received in the cutout in an orientation relative to an elongated body of the catheter substantially the same as when the dual lumen tip and the elongated body of the catheter are in an unstressed state.

2. The tunneler according to claim 1, further comprising a sheath extendable around the catheter-coupling end to encase a dual lumen tip of a catheter coupled to the tunneler.

3. The tunneler according to claim 1, wherein the cutout includes a substantially flat tip receiving surface.

4. The tunneler according to claim 1, wherein the cutout includes a tip receiving surface contoured to complement a shape of a radially inward surface of a dual lumen tip catheter to be coupled thereto.

5. The tunneler according to claim 1, wherein the lumen-mating projection includes a barb to anchor the lumen-mating projection within a lumen of a catheter to be coupled to the tunneler.

6. The tunneler according to claim 1, further comprising a lip extending proximally over a portion of the cutout to limit radially outward movement of a dual lumen tip received therein.

7. The tunneler according to claim 6, wherein the barb has a shaft with a length substantially equal to a length of the dual lumen tip.

8. The tunneler according to claim 1, wherein the barb is offset from a centerline of the tunneler, opposite to the dual lumen tip.

9. The tunneler according to claim 8, wherein the projection is offset from an axis of the tunneler by a distance substantially equal to an offset of the dual lumen tip from an axis of an elongate body of a catheter to be coupled thereto.

10. The tunneler according to claim 1, wherein the tunneler is formed of one of a metal and a polymer.

11. The tunneler according to claim 1, wherein the catheter coupling end flares outward to smoothly transition from a diameter of the elongated body to an increased proximal diameter.

12. The tunneler according to claim 11, wherein the increased proximal diameter is selected to be at least as great as a maximum distance between a radially outer surface of a dual lumen tip of a catheter to be coupled thereto and an opposite radially outer surface of an elongate body of the catheter.

13. The tunneler according to claim 2, wherein the sheath is slidably received around the tunneler.

14. The tunneler according to claim 13, wherein an opening extends through a distal end of the sheath for receiving the elongated body therethrough, a diameter of the opening be less than a maximum diameter of the catheter coupling end.

15. The tunneler according to claim 2, wherein the sheath is permanently coupled to the catheter coupling end.

16. The tunneler according to claim 1, wherein the lumen-mating projection is radially offset from a centerline of the elongated body.

17. The tunneler according to claim 10, wherein the metal is stainless steel.

18. The tunneler according to claim 10, wherein the polymer is Delrin.

19. The tunneler according to claim 1, wherein the cutout is sized and shaped so that, when received therein, a radially outermost portion of the dual lumen tip is within a maximum diameter of the catheter tip receiving portion.

20. The tunneler according to claim 2, wherein a diameter of the sheath is selected to apply a force to the catheter to maintain the lumen-mating projection in an interference fit within a catheter coupled to the tunneler.
